SEQUENCE LISTING (6 sequences)

SEQ ID NO: 1 - DNA, 1269 bp, Homo sapiens
  CDS (1)..(1269)
  misc_structure (418)..(810): C2A domain
atggtgagcgagagtcaccatgaggccctggcagccccgcctgtcaccactgtcgcgactgttctgccaagcaatgccacagagccagccagtcctggagaaggaaaggaagatgcattttctaagctgaaggagaagtttatgaatgagttgcataaaattccattgccaccgtgggccttaattgcaatagccatagtcgcagtccttttagtcctgacctgctgcttttgtatctgtaagaaatgtttgttcaaaaagaaaaacaagaagaagggaaaggaaaaaggagggaagaatgccattaacatgaaagatgtaaaagacttagggaagacgatgaaagatcaggccctcaaggatgatgatgctgaaactggattgacagatggagaagaaaaagaagaacccaaagaagaggagaaactgggaaaacttcagtattcactggattatgatttccaaaataaccagctgctggtagggatcattcaggctgctgaactgcccgccttggacatggggggcacatctgatccttacgtgaaagtgtttctgctacctgataagaagaagaaatttgagacaaaagtccaccgaaaaacccttaatcctgtcttcaatgagcaatttactttcaaggtaccatactcggaattgggtggcaaaaccctagtgatggctgtatatgattttgatcgtttctctaagcatgacatcattggagaatttaaagtccctatgaacacagtggattttggccatgtaactgaggaatggcgtgacctgcaaagtgctgagaaggaagagcaagagaaattgggtgatatctgcttctcccttcgctacgtacctactgctggtaagctgactgttgtcattctggaggcaaagaacctgaagaagatggatgtgggtggcttatccgatccttatgtgaagattcatctgatgcagaatggtaagaggctgaagaagaaaaagacaacaattaaaaagaacacacttaacccctactacaatgagtcattcagctttgaagtaccttttgaacaaatccagaaagtgcaggtggtggtaactgttttggactatgacaagattggcaagaacgatgccatcggcaaagtctttgtgggctacaacagcaccggcgcggagctgcgacactggtcagacatgctggccaaccccaggcgacctattgcccagtggcacaccctgcaggtagaggaggaagttgatgccatgctggccgtcaagaagtaa

SEQ ID NO: 2 - protein, 422 aa, Homo sapiens
MVSESHHEALAAPPVTTVATVLPSNATEPASPGEGKEDAFSKLKEKFMNELHKIPLPPWALIAIAIVAVLLVLTCCFCICKKCLFKKKNKKKGKEKGGKNAINMKDVKDLGKTMKDQALKDDDAETGLTDGEEKEEPKEEEKLGKLQYSLDYDFQNNQLLVGIIQAAELPALDMGGTSDPYVKVFLLPDKKKKFETKVHRKTLNPVFNEQFTFKVPYSELGGKTLVMAVYDFDRFSKHDIIGEFKVPMNTVDFGHVTEEWRDLQSAEKEEQEKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIAQWHTLQVEEEVDAMLAVKK

SEQ ID NO: 3 - DNA, 1266 bp, Rattus norvegicus
  CDS (1)..(1266)
  misc_structure (418)..(798): C2A domain
atggtgagtgccagtcatcctgaggccctggccgcccctgtcaccactgttgcgacccttgtcccacacaatgccactgagccagccagtcctggggaagggaaggaagatgccttttccaagctgaagcagaagtttatgaatgagctgcataaaattccattgccaccgtgggccttaatagccatagccatagttgcggtccttttagtcgtaacctgctgcttttgtgtctgtaagaaatgtttgttcaaaaagaaaaacaagaagaaggggaaggaaaagggaggaaagaacgccattaacatgaaagacgtgaaagacttagggaagaccatgaaggatcaggcccttaaggatgacgatgctgaaaccggactgactgatggagaagaaaaggaagagcccaaggaagaggagaaactgggaaagctccaatattcactggactatgacttccagaataaccagctgttggtgggaatcatccaggctgctgaactgcccgccctggacatggggggtacatccgatccatacgtcaaagtcttcctgctgcctgaaaaaaagaagaaatttgagactaaagtccaccggaaaaccctcaatccagtcttcaatgaacaatttactttcaaggtaccctactcggaattaggtggcaaaaccctggtgatggctgtgtatgactttgatcgcttctccaagcacgacatcatcggagagttcaaagttcctatgaacaccgtggattttggccatgtgaccgaggagtggcgcgatctccagagcgctgagaaagaagagcaagagaaactgggtgacatctgcttctccctccgctacgtccctactgccggcaaactgactgttgtcattctggaagccaagaacctgaagaagatggatgtgggtggcttatctgatccctacgtgaagattcacctgatgcagaacggtaagaggctgaagaagaaaaagacgacgattaagaagaacacactcaacccctactacaacgagtccttcagctttgaagttccgttcgagcaaatccagaaagtgcaagtggtggtaactgttttggactatgacaagattggcaagaacgacgccatcgacaaagtcttcgttggttacaacagcactggggcggagctgcgacactggtcagacatcctggccaacccccggcgacccatcgcacagtggcacactctgcaggtagaggaggaggttgatgccatgctggctgtcaagaagtaa

SEQ ID NO: 4 - protein, 421 aa, Rattus norvegicus
MVSASHPEALAAPVTTVATLVPHNATEPASPGEGKEDAFSKLKQKFMNELHKIPLPPWALIAIAIVAVLLVVTCCFCVCKKCLFKKKNKKKGKEKGGKNAINMKDVKDLGKTMKDQALKDDDAETGLTDGEEKEEPKEEEKLGKLQYSLDYDFQNNQLLVGIIQAAELPALDMGGTSDPYVKVFLLPEKKKKFETKVHRKTLNPVFNEQFTFKVPYSELGGKTLVMAVYDFDRFSKHDIIGEFKVPMNTVDFGHVTEEWRDLQSAEKEEQEKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIDKVFVGYNSTGAELRHWSDILANPRRPIAQWHTLQVEEEVDAMLAVKK

SEQ ID NO: 5 - DNA, 1266 bp, Mus musculus
  CDS (1)..(1266)
  misc_structure (418)..(810): C2A domain
atggtgagtgccagtcgtcctgaggccctggctgcccctgtcaccactgttgcgacccttgtcccacacaacgccactgagccagccagtcctggggaagggaaggaagatgccttttccaagctgaagcagaagtttatgaatgaactgcataaaatcccattgccaccgtgggccttaattgccatagccatagttgcggtccttctagtcgtgacctgctgcttctgtgtctgtaagaaatgtttgttcaaaaagaaaaacaagaagaagggaaaggaaaagggagggaagaacgccattaacatgaaagacgtgaaagacttagggaagaccatgaaggatcaggcccttaaggatgacgatgctgaaactggactgactgatggagaagaaaaggaggagcccaaggaagaggagaaactgggaaagcttcaatattcactggactatgacttccagaataaccagctgctggtgggaatcatccaggctgctgaactgcccgccctggacatgggaggcacatctgatccatacgtcaaagtcttcctgctgcccgacaaaaagaagaagtttgagacaaaagtccaccggaaaaccctcaatccagtcttcaatgaacagtttactttcaaggtgccatactcggaattaggtggcaagacactggtgatggctgtgtatgattttgaccgcttctccaagcacgacatcattggagagttcaaagttcctatgaacaccgtggattttggccacgtcaccgaggagtggcgcgatctccagagtgctgagaaagaagagcaagagaaactgggtgacatctgcttctccctccgctacgtccctactgccggcaagctgactgttgtcattctggaagccaagaacctgaagaagatggatgtgggtggcttatctgatccctatgtaaagattcacctgatgcagaacggcaagagactgaagaagaaaaagacaacgattaagaagaacacacttaacccctactacaatgagtccttcagctttgaagttccgttcgagcaaatccagaaagtgcaagtggtggtaactgttttggactatgacaagattggcaagaacgacgccatcggcaaagtctttgtgggctacaacagcaccggcgcagagctgcgacactggtcagacatgctggccaacccccggcgacccatcgcccagtggcacactctgcaggtagaggaggaggttgatgccatgctggctgtcaagaagtaa

SEQ ID NO: 6 - protein, 421 aa, Mus musculus
MVSASRPEALAAPVTTVATLVPHNATEPASPGEGKEDAFSKLKQKFMNELHKIPLPPWALIAIAIVAVLLVVTCCFCVCKKCLFKKKNKKKGKEKGGKNAINMKDVKDLGKTMKDQALKDDDAETGLTDGEEKEEPKEEEKLGKLQYSLDYDFQNNQLLVGIIQAAELPALDMGGTSDPYVKVFLLPDKKKKFETKVHRKTLNPVFNEQFTFKVPYSELGGKTLVMAVYDFDRFSKHDIIGEFKVPMNTVDFGHVTEEWRDLQSAEKEEQEKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIAQWHTLQVEEEVDAMLAVKK

(19) United States
(12) Patent Application Publication
(10) Pub. No.: US 2006/0110323 A1
(43) Pub. Date: May 25, 2006

(54) Method of imaging cell death in vivo

(76) Inventor: Ming Zhao, Brookfield, WI (US)

Correspondence Address: QUARLES & BRADY LLP, 411 E. WISCONSIN AVENUE, SUITE 2040, MILWAUKEE, WI, 53202-4497, US

(21) Appl. No.: 11/281,957

(22) Filed: Nov. 17, 2005

Related U.S. Application Data
(60) Provisional application No. 60/629,607, filed on Nov. 19, 2004.

(51) Int. Cl. A61K 51/00

(52) U.S. Cl. 424/1.11

(57) ABSTRACT

The present invention relates to a method and a kit for detecting cell death or another condition characterized by an increase in the extracellular level of phosphatidylserine in a mammalian subject. The method involves administering a radionuclide-labeled compound that comprises a C2 domain of a protein or an active variant thereof and measuring radiation emission from the radionuclide in the subject to obtain an image of radiation emission, wherein the site of cell death or said condition can be determined from the image. The kit can contain a radionuclide-labeled compound comprising a C2 domain or an active variant thereof and an instruction on administering the compound into a mammalian subject to image cell death or said condition.

### CROSS-REFERENCE TO RELATED APPLICATIONS

This application claims the benefit of U.S. provisional patent application Serial No. 60/629,607, filed on Nov. 19, 2004, which is herein incorporated by reference in its entirety.

### STATEMENT REGARDING FEDERALLY SPONSORED RESEARCH OR DEVELOPMENT

Not applicable.

### BACKGROUND OF THE INVENTION

Non-invasive imaging of cell death has important diagnostic and prognostic predictive potentials. Collectively, the pathological causes to structural and functional loss to otherwise healthy tissues may be attributed to an interplay of different modes of cell death. On the other hand, tumoral cell death after therapeutic treatments has a positive correlation with patient survival (Kostin S. et al. Circ. Res. 92:715-24, 2003; and Selivanova G. Current Cancer Drug Targets 4:385-402, 2004). Among the two dominant forms of cell death, apoptosis has gained much attention in modem medicine not only because of the deleterious consequences that its deregulation can have, but also as an opportunity for therapeutic intervention (Kerr J F et al. Br J Cancer 26:239-57, 1972; Kerr, J. F. Toxicology. 181-182, 471-4, 2002; Huang P. et al. Curr Opin Oncol 9:94-100, 1997; and Gourley M et al. Curr Pharm Des 6:417-39, 2000). In contrast to necrosis, which has a passive nature characterized by plasma membrane rupture, apoptosis is an intracellular energy-dependent process (Song Z et al. Trends. Cell. Biol. 9:M49-52, 1999; and Wyllie A H Br Med Bull 53:451-65, 1997). Once committed, the execution phase of apoptosis involves a proteolytic cascade catalyzed by caspases and is accompanied by the appearance of distinct molecular markers (Grutter M G Curr Opin Struct Biol 10:649-55, 2000; Green D R Cell 94:695-98, 1998; Thomberry N A et al. Science 281:1312-16, 1998; and Cohen G M. Biochem J 326:1-16, 1997).

A common molecular marker for both apoptosis and necrosis as well as other types of cell death is the exposure of phosphatidylserine (PtdS), and its identification can facilitate target-specific imaging of cell death. In viable cells, PtdS is a component of the inner leaflet of the plasma membrane, and virtually absent on the cell surface. The asymmetry of the lipid bilayer is maintained by the actions of energy dependent enzymes, including aminophospholipid translocase and floppase (Williamson P et al. Biochim Biophys Acta 1585:53-63, 2002). During apoptosis, the inhibition of translocase and floppase is accompanied by the activation of scramblase, and the redistribution of phospholipids across the bilayers is facilitated (Williamson P et al. Biochim Biophys Acta 1585:53-63, 2002). As a result, PtdS becomes exposed onto the cell surface. In necrotic cells, however, the exposure of PtdS is a rather passive incidence, due to rupture of the plasma membrane that renders intracellular components accessible to extracellular environment. Being one of the major phospholipid components of the plasma membrane, PtdS provides an abundant molecular marker once it becomes accessible.

Annexin V binds to PtdS with high affinity. Annexin V and its analogues labeled with a chromogen or radionuclide (e.g., technetium 99 or ^{99m}Tc) have been used to identify apoptotic cells both in vitro and in vivo (see e.g., Blankenberg et al. Proc. Natl. Acad. Sci. U.S.A. 95:6349-6354, 1998; Vriens et al. J. Thorac. Cardiovasc. Surg. 116:844-853, 1998; Ohtsuki K et al. Eur J Nucl Med 26:1251-58, 1999; Petrovsky A et al. Cancer Res 63:1936-42, 2003; and Lahorte CMM et al. Eur J Nucl Med 31:887-919, 2004). In particular, radionuclide-labeled annexin V has been used to detect myocardial cell death in human patients (Hosstra L et al. Lancet 356:209-12, 2000). However, successful detection could not be made till 17 to 22 hour post injection, rendering it impractical for use with patients who suffer acute infarction in the heart.

Another protein, the C2A domain of synaptotagmin I, has been shown to recognize both necrotic and apoptotic cells by binding to exposed PtdS in a calcium-dependent manner (Davletov B A et al. J Biol Chem 268:26386-90, 1993). The C2A domain of synaptotagmin I labeled with fluorochromes or superparamagnetic nanoparticles has allowed detection of cell death using fluorescent or magnetic resonance imaging techniques, respectively (Zhao M et al. Nat. Med. 7:1241-1244, 2001; Jung H I et al. Bioconjugate Chem 15:983-7, 2004; and U.S. Patent Application Publication 2004/0022731). However, the feasibility of using a radionuclide-labeled C2 domain for imaging cell death, especially within the early hours after the onset of a disease or condition such as acute ischemia and reperfusion, is not clear.

### BRIEF SUMMARY OF THE INVENTION

The present invention relates to a method and a kit for detecting cell death or another condition characterized by an increase in the extracellular level of PtdS in a mammalian subject. The method involves administering a radionuclide-labeled compound that comprises a C2 domain of a protein or an active variant thereof and measuring radiation emission from the radionuclide in the subject to obtain an image of radiation emission, wherein the site of cell death or said condition can be determined from the image. The kit can contain a radionuclide-labeled compound comprising a C2 domain or an active variant thereof and an instruction on administering the compound into a mammalian subject to image cell death or said condition.

### BRIEF DESCRIPTION OF THE SEVERAL VIEWS OF THE DRAWINGS

FIG. 1 shows flow cytometry analysis of camptothecin treated Jurkat cells. Double labeling using propidium iodide (PI) and C2A-GST-FITC or Annexin V-FITC is shown in 1a. Dual probe labeling with C2A-GST-AF680 and Annexin V-FITC is shown in 1b.

FIG. 2 shows elution profile of ^{99m}Tc-C2A-GST from G-25 sephadex gel filtration column chromatography, in terms of radioactivity (solid circles) obtained by gamma counting, and relative protein concentration (open squares) measured in absorbance at 512 nm after staining with Bradford method.

FIG. 3 shows dissociation constant (Kd) measurement using a saturation method for ^{99m}Tc-C2A-GST using camptothecin treated Jurkat cells. The Kd is determined as the concentration of ^{99m}Tc-C2A-GST at half (B_{1/2}) of maximal binding (Bₘₐₓ).

FIG. 4 shows competition assay with ^{99m}Tc-C2A-GST against unlabeled C2A-GST. The half inhibitory concentration (IC₅₀) is determined as the concentration of the unlabeled C2A-GST where half of the bound radioactivity is displaced.

FIG. 5 shows biodistribution of ^{99m}Tc-C2A-GST in mice (n=8 for each time point) at 1, 15, 30, 60, 120 and 240 min after tail vein injection. The percentage of injected dosage of each organ is presented over time.

FIG. 6 shows flow cytometry of cardiac cells taken from the infarct and remote viable region after SPECT imaging. The cells were sorted based on the relative DNA content, as stained using propidium iodide.

FIG. 7 shows histological analysis of cardiac tissues taken from the infarct site, demonstrating classical ultrastructural changes associated with acute myocardial infarction. Transmission electron microscopy is shown at the top, and H&E staining of tissue sections are shown at the bottom. Chromatin condensation/marginalization, mitochondrial abnormality, and myofibril hyper-contraction are marked by asterisks, arrows, and arrow heads, respectively.

### DETAILED DESCRIPTION OF THE INVENTION

The present invention relates to a non-invasive method of imaging or detecting cell death or another condition characterized by an increase in the extracellular level of PtdS in a mammalian subject. The method involves administering to the subject an effective amount of a radionuclide-labeled compound, preferably a radionuclide-labeled polypeptide, that comprises a C2 domain of a protein or an active variant of said C2 domain and measuring radiation emission from the radionuclide in the subject to obtain an image of radiation emission. The site of cell death or said condition can be determined from the image. Radiation emission from the subject can be measured more than once at selected intervals to track changes in emission intensity over time so that changes such as in the number or distribution of cells that undergo cell death can be determined.

Apoptosis, necrosis, and other types of cell death as well as other conditions characterized by an increase in the extracellular level of PtdS can be detected by the method of the present invention. The present invention is especially useful for detecting heart infarction, vascular thrombi, and atherosclerotic plaques, for example in mammalian subjects suspected of having one of these conditions, as these conditions have been shown to be associated with increased level of PtdS for extracellular binding. Heart infarction is characterized by necrosis of the heart tissue as a result of obstruction of local blood supply, as by a thrombus or an embolus. Vascular thrombi contain activated platelets that express a significantly greater amount of PtdS than quiescent platelets, which express little, if any PtdS. For atherosclerotic plaques, a significant proportion of the cells undergo cell death (see e.g., Crisby M et al. Atherosclerosis 130:17-27, 1997).

The examples below demonstrate that the use of a radionuclide-labeled C2-domain allows image acquisition at a much earlier time point post injection than a radionuclide-labeled annexin V does, making the former an advantageous imaging agent over the latter, especially for diseases and conditions the successful treatment of which depends on timely diagnosis (e.g., acute heart infarction). Existing apoptosis imaging techniques with radiolabeled annexin V allow image acquisition only after 15 to 22 hours post injection, due to the relatively slow clearance of the radio tracer. At about 3 times the radioactive half-life of ^{99m}Tc, such imaging protocol requires the administration of high radiation dosages, and prolonged patient waiting time. In contrast, it is demonstrated here (example 2 below) that radiolabeled C2 domain allows image acquisition at a much earlier time point. This was confirmed by postmortem analysis, including scintillation counting, flow cytometry, and electron microscopy.

The method of the present invention can also be used for imaging tumor cell death in a mammalian animal (e.g., a cancer patient) undergoing treatment designed to cause cell death in the tumor (e.g., chemotherapy), thereby providing information on whether the treatment is likely to be successful.

In a preferred embodiment, the present invention is practiced with a mammalian subject that is a pig, a rat, or a mouse. In a more preferred embodiment, the mammalian subject is a human. Cell death or another condition characterized by an increase in the extracellular level of PtdS may be imaged or detected in, for example, an organ or tumor of a mammalian subject or a portion thereof.

C2 domains from various proteins are well known in the art. While some of the proteins (e.g., protein kinase C alpha) have only one C2 domain, others (e.g., synaptotagmin I) have two or more. For a protein that contains two or more C2 domains, the domains are conveniently distinguished in the art by attaching a letter (in alphabetical order) to the end of the name (e.g., C2A, C2B, and so on). For a protein that contains only one C2 domain, the domain is simply referred to as C2 domain. For the purpose of the present invention, the term “C2 domain” is used to encompass all C2 domains, regardless whether they are the only C2 domain or one of the multiple C2 domains on a protein. While the examples below use the C2A domain of rat synaptotagmin I as the representative to demonstrate the present invention, all C2 domains with PtdS binding activity can be used for the purpose of the present invention. A common structural feature shared by all C2 domains is the eight stranded antiparallel β-sandwich connected by variable loops (Brose N. et al. J. Biol. Chem. 270:25273-80, 1995; Davletov B. A. et al. J. Biol. Chem. 268:26386-90, 1993; Rizo J. et al. J. Biol. Chem. 273:15879-15882, 1998; Sutton R. B. et al. Cell 80:929-38, 1995, all of which are herein incorporated by reference in their entirety). Examples of proteins that contain a C2 domain include but are not limited to synaptotagmin 1-13, protein kinase C family members of serine/threonine kinases, phospholipase A2, phospholipase δ1, cofactors in the coagulation cascade including factors V and VIII, and members of the copine family. Human synaptotagmins include synaptotagmin 1-7, 12, and 13.

Some specific examples of the proteins that contain a C2 domain includes but not limited to (Swiss-Prot/TrEMBL entry names followed by accession numbers in parentheses): ABR_HUMAN (Q12979), BAIP3_HUMAN (O94812), BAIP3_MOUSE (Q80TT2), BCR_HUMAN (P11274), BUD2_YEAST (P33314), CPNE1_HUMAN (Q99829), CPNE1_MOUSE (Q8C166), CPNE2_HUMAN (Q96FN4), CPNE2_MOUSE (P59108), CPNE3_HUMAN (O75131), CPNE3_MOUSE (Q8BT60), CPNE3_PONPY (Q5RAE1), CPNE4_HUMAN (Q96A23), CPNE4_MOUSE (Q8BLR2), CPNE5_HUMAN (Q9HCH3), CPNE5_MOUSE (Q8JZW4), CPNE6_HUMAN (O95741), CPNE6_MOUSE (Q9Z140), CPNE6_PONPY (Q5R4W6), CPNE7_HUMAN (Q9UBL6), CPNE8_HUMAN (Q86YQ8), CPNE8_MOUSE (Q9DC53), DOC2A_HUMAN (Q14183), DOC2A_MOUSE (Q7TNF0), DOC2A_RAT (P70611), DOC2B_HUMAN (Q14184), DOC2B_MOUSE (P70169), DOC2B_RAT (P70610), DOC2G_MOUSE (Q9ESN1), DYSF_HUMAN (O75923), DYSF_MOUSE (Q9ESD7), ERG1_ORYSA (Q7GC09), ERG3_ORYSA (Q7F9X0), FER1_CAEEL (Q17388), GAP1_DROME (P48423), ITCH_HUMAN (Q96J02), ITCH_MOUSE (Q8C863), ITSN1_HUMAN (Q15811), ITSN1_MOUSE (Q9Z0R4), ITSN2_HUMAN (Q9NZM3), ITSN2_MOUSE (Q9Z0R6), KPC1B_CAEEL (P34885), KPC1_APLCA (Q16974), KPC1_DROME (P05130), KPC1_LYTPI (Q25378), KPC2_APLCA (Q16975), KPC2_CAEEL (P90980), KPC2_DROME (P13677), KPCA_BOVIN (P04409), KPCA_HUMAN (P17252), KPCA_MOUSE (P20444), KPCA_RABIT (P10102), KPCA_RAT (P05696), KPCB_BOVIN (P05126), KPCB_HUMAN (P05771), KPCB_MOUSE (P68404), KPCB_RABIT (P05772), KPCB_RAT (P68403), KPCE_HUMAN (Q02156), KPCE_MOUSE (P16054), KPCE_RABIT (P10830), KPCE_RAT (P09216), KPCG_BOVIN (P05128), KPCG_HUMAN (P05129), KPCG_MOUSE (P63318), KPCG_RABIT (P10829), KPCG_RAT (P63319), KPCL_HUMAN (P24723), KPCL_MOUSE (P23298), KPCL_RAT (Q64617), MYOF_HUMAN (Q9NZM1), NED4L_HUMAN (Q96PU5), NED4L_MOUSE (Q8CFI0), NED4L_PONPY (Q5RBF2), NEDD4_DROME (Q9VVI3), NEDD4_HUMAN (P46934), NEDD4_MOUSE (P46935), NEDD4_RAT (Q62940), OTOF_HUMAN (Q9HC10), OTOF_MOUSE (Q9ESF1), P3C2A_HUMAN (O00443), P3C2A_MOUSE (Q61194), P3C2A_PONPY (Q5RAY1), P3C2B_HUMAN (O00750), PA24A_BRARE (P50392), PA24A_CHICK (P49147), PA24A_HORSE (O77793), PA24A_HUMAN (P47712), PA24A_MOUSE (P47713), PA24A_RAT (P50393), PCLO_CHICK (Q9PU36), PCLO_HUMAN (Q9Y6V0), PCLO_MOUSE (Q9QYX7), PCLO_RAT (Q9JKS6), PERF_HUMAN (P14222), PERF_MOUSE (P10820), PERF_RAT (P35763), PIP1_DROME (P25455), PIPA_DICDI (Q02158), PIPA_DROME (P13217), PLC1_SCHPO (P40977), PLC1_YEAST (P32383), PLCB1_BOVIN (P10894), PLCB1_HUMAN (Q9NQ66), PLCB1_MOUSE (Q9Z1B3), PLCB1_RAT (P10687), PLCB2_HUMAN (Q00722), PLCB2_RAT (089040), PLCB3_HUMAN (Q01970), PLCB3_MOUSE (P51432), PLCB3_RAT (Q99JE6), PLCB4_BOVIN (Q07722), PLCB4_HUMAN (Q15147), PLCB4_RAT (Q9QW07), PLCD1_BOVIN (P10895), PLCD1_HUMAN (P51178), PLCD1_MOUSE (Q8R3B1), PLCD1_RAT (P10688), PLCG1_BOVIN (P08487), PLCG1_HUMAN (P19174), PLCG1_RAT (P10686), PLCG2_HUMAN (P16885), PLCG2_RAT (P24135), PLCL4_HUMAN (O75038), PLDA1_PIMBR (O04883), PLDB1_ARATH (P93733), PLDB2_ARATH (O23078), PLDD1_ARATH (Q9C5Y0), PLDG1_ARATH (Q9T053), PLDG2_ARATH (Q9T051), PLDG3_ARATH (Q9T052), PP16A_CUCMA (Q9ZT47), PP16B_CUCMA (Q9ZT46), PUB1_SCHPO (Q92462), PUB3_SCHPO (O14326), RASA1_BOVIN (P09851), RASA1_HUMAN (P20936), RASA1_RAT (P50904), RASA2_HUMAN (Q15283), RASA2_MOUSE (P58069), RASA2_RAT (Q63713), RASA3_BOVIN (Q28013), RASA3_HUMAN (Q14644), RASA3_MOUSE (Q60790), RASL1_HUMAN (O95294), RASL1_MOUSE (Q9Z268), RASL2_HUMAN (O43374), RBF1_CAEEL (P41885), RFIP1_HUMAN (Q6WKZ4), RFIP1_MOUSE (Q9D620), RFIP2_HUMAN (Q7L804), RFIP5_HUMAN (Q9BXF6), RFIP5_MOUSE (Q8R361), RGS3_HUMAN (P49796), RIMS1_HUMAN (Q86UR5), RIMS1_RAT (Q9JIR4), RIMS2_HUMAN (Q9UQ26), RIMS2_MOUSE (Q9EQZ7), RIMS2_RAT (Q9JIS1), RIMS3_HUMAN (Q9UJD0), RIMS3_MOUSE (Q80U57), RIMS3_RAT (Q9JIR3), RIMS4_HUMAN (Q9H426), RIMS4_MOUSE (P60191), RIMS4_RAT (Q8CIX1), RIM_CAEEL (Q22366), RP3A_BOVIN (Q06846), RP3A_HUMAN (Q9Y2J0), RP3A_MOUSE (P47708), RP3A_RAT (P47709), RPGR1_BOVIN (Q9GLM3), RPGR1_MOUSE (Q9EPQ2), RSP5_YEAST (P39940), SMUF1_DROME (Q9V853), SMUF1_HUMAN (Q9HCE7), SMUF1XENLA (Q9PUN2), SMUF2_HUMAN (Q9HAU4), SUDX_DROME (Q9Y0H4), SY61_DISOM (P24505), SY62_DISOM (P24506), SY63_DISOM (P24507), SY65_APLCA (P41823), SY65_DROME (P21521), SYT10_HUMAN (Q6XYQ8), SYT10_MOUSE (Q9R0N4), SYT10_PONPY (Q5RCK6), SYT10_RAT (O08625), SYT11_HUMAN (Q9BT88), SYT11_MOUSE (Q9R0N3), SYT11_RAT (O08835), SYT12_HUMAN (Q8IV01), SYT12_MOUSE (Q920N7), SYT12_RAT (P97610), SYT14_HUMAN (Q8NB59), SYT14_MOUSE (Q7TN84), SYT15_HUMAN (Q9BQS2), SYT15_MOUSE (Q8C6N3), SYT15_RAT (P59926), SYT1_BOVIN (P48018), SYT1_CAEEL (P34693), SYT1_CHICK (P47191), SYT1_HUMAN (P21579), SYT1_MACFA (Q60HC0), SYT1_MOUSE (P46096), SYT1_PONPY (Q5R4J5), SYT1_RAT (P21707), SYT2_HUMAN (Q8N9I0), SYT2_MOUSE (P46097), SYT2_RAT (P29101), SYT3_HUMAN (Q9BQG1), SYT3_MOUSE (O35681), SYT3_RAT (P40748), SYT4_HUMAN (Q9H2B2), SYT4_MOUSE (P40749), SYT4_RAT (P50232), SYT5_HUMAN (O00445), SYT5_MOUSE (Q9R0N5), SYT5_RAT (P47861), SYT6_HUMAN (Q5T7P8), SYT6_MOUSE (Q9R0N8), SYT6_RAT (Q62746), SYT7_HUMAN (O43581), SYT7_MOUSE (Q9R0N7), SYT8_MOUSE (Q9R0N6), SYT8_RAT (Q925B4), SYT9_HUMAN (Q86SS6), SYT9_MOUSE (Q9R0N9), SYT9_RAT (Q925C0), SYTL1_HUMAN (Q8IYJ3), SYTL1_MOUSE (Q99N80), SYTL2_HUMAN (Q9HCH5), SYTL2_MOUSE (Q99N50), SYTL3_MOUSE (Q99N48), SYTL4_HUMAN (Q96C24), SYTL4_MOUSE (Q9R0Q1), SYTL4_RAT (Q8VHQ7), SYTL5_HUMAN (Q8TDW5), SYTL5_MOUSE (Q80T23), SYTL5_RAT (Q812E4), TAC2N_HUMAN (Q8N9U0), TAC2N_MOUSE (Q91XT6), UN13A_HUMAN (Q9UPW8), UN13A_RAT (Q62768), UN13B_HUMAN (O14795), UN13B_MOUSE (Q9Z1N9), UN13B_RAT (Q62769), UN13C_HUMAN (Q8NB66), UN13C_MOUSE (Q8K0T7), UN13C_RAT (Q62770), UN13D_HUMAN (Q70J99), UN13D_RAT (Q9R189), UNC13_CAEEL (P27715), WWP1_HUMAN (Q9H0M0), WWP1_MOUSE (Q8BZZ3), Y1322_ARATH (Q9C8S6), YC31_SCHPO (O14065), YGJI_CAEEL (Q9XUB9), YKH3_SCHPO (Q9UT00), YMH2_YEAST (Q03640), and YNI7_YEAST (P48231). See us.expasy.org/cgi-bin/prosite-search-ac?PS50004 at the ExPASy (Expert Protein Analysis System) proteomics server of the Swiss Institute of Bioinformatics (SIB).

Some other specific examples of the proteins that contain a C2 domain includes but not limited to (Swiss-Prot/TrEMBL entry names followed by accession numbers in parentheses): CAN5_CAEEL (Q22036), CAN5_HUMAN (O15484), CAN5_MOUSE (O08688), CAN5_RAT (Q8R4C0), CAN6_HUMAN (Q9Y6Q1), CAN6_MOUSE (O35646), CAN6_RAT (O88501), GAP2_CAEEL (Q8MLZ5), GAP2_DROME (Q8T498), INP4A_HUMAN (Q96PE3), INP4A_MOUSE (Q9EPW0), INP4A_RAT (Q62784), INP4B_HUMAN (O15327), INP4B_MACFA (Q4R4D7), INP4B_MOUSE (Q6P1Y8), INP4B_PONPY (Q5RA60), INP4B_RAT (Q9QWG5), KPC1_YEAST (P24583), KPCD_CANFA (Q5PU49), KPCD_HUMAN (Q05655), KPCD_MOUSE (P28867), KPCD_RAT (P09215), NGAP_HUMAN (Q9UJF2), P3C2G_HUMAN (O75747), P3C2G_MOUSE (O70167), P3C2G_RAT (O70173), PI3K1_SOYBN (P42347), PI3K2_SOYBN (P42348), PK3CA_BOVIN (P32871), PK3CA_HUMAN (P42336), PK3CA_MOUSE (P42337), PKL1_HUMAN (Q16512), PKL1_MOUSE (P70268), PKL1_RAT (Q63433), PKL2_HUMAN (Q16513), PKL2_MOUSE (Q8BWW9), PKL2_RAT (O08874), PLC1_CANAL (O13433), PLDA1_ARATH (Q38882), PLDA1_BRAOC (O82549), PLDA1_MAIZE (Q43270), PLDA1_ORYSA (Q43007), PLDA1_RICCO (Q41142), PLDA1_TOBAC (P93400), PLDA1_VIGUN (O04865), PLDA2_ARATH (Q9SSQ9), PLDA2_BRAOC (P55939), PLDA2_ORYSA (P93844), PLDE1_ARATH (Q9C888), PLDZ1_ARATH (P58766), PUB2_SCHPO (Q9UTG2), RGS7_CAEEL (Q8WQC0), RPGR1_HUMAN (Q96KN7), SCH9_YEAST (P11792), SYGP1_HUMAN (Q96PV0), SYGP1_RAT (Q9QUH6), UVRAG_HUMAN (Q9P2Y5), WWP2_HUMAN (O00308), and WWP2_MOUSE (Q9DBH0). See us.expasy.org/cgi-bin/prosite-search-ac?PS50004 at the ExPASy (Expert Protein Analysis System) proteomics server of the Swiss Institute of Bioinformatics (SIB).

In a preferred embodiment, a synaptotagmin I C2A domain from human, rat, or mouse is used to practice the invention. The cDNA and amino acid sequences for human synaptotagmin I (the cDNA sequence is set forth in SEQ ID NO:1 and the amino acid sequence is set forth in SEQ ID NO:2), rat synaptotagmin I (the cDNA sequence is set forth in SEQ ID NO:3 and the amino acid sequence is set forth in SEQ ID NO:4), and mouse synaptotagmin I (the cDNA sequence is set forth in SEQ ID NO:5 and the amino acid sequence is set forth in SEQ ID NO:6) can be found at GenBank Accession Nos. M55047, NM_{—}001033680, and NM_{—}009306, respectively. For human, rat, and mouse synaptotagmin I, the C2A domain spans amino acids 140-270, amino acids 140-266, and amino acids 140-270, respectively.

As used herein, an active variant of a wild-type C2 domain refers to a polypeptide that differs from said particular wild-type C2 domain by one or more residues (e.g., by deletion, insertion, or substitution), but is at least 60%, 65%, 70%, 75%, 80%, 85%, 90%, 95%, 97%, or 99% identical to said particular wild-type C2 domain over the full length of said wild-type C2 domain, and has specific PtdS binding activity. While in many cases, an active variant of a wild-type C2 domain is not a naturally occurring peptide, it can be another wild-type C2 domain from a different protein or from the corresponding protein of a different species. When an active variant is a fragment of a wild-type C2 domain, it is preferred that the fragment is at least 80%, 85%, 90%, 95%, 97% or 99% of the length of said wild-type domain. In a preferred embodiment, an active variant of a wild-type C2 domain is of the same length of said wild-type C2 domain. In another preferred embodiment, an active variant is a wild-type C2 domain with one or more conservative substitutions. It is well known in the art that the amino acids within the same conservative group can typically substitute for one another without substantially affecting the function of a protein. For the purpose of the present invention, such conservative groups are set forth in Table 1 based on shared properties.

TABLE 1
Conservative substitution.

| Original Residue | Conservative Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Thr, Ser |
| Val (V) | Ile, Leu, Met, Phe, Ala |

In another embodiment, a C2 domain or an active variant thereof has a peptide tag such a GST tag attached for facilitating protein isolation or other purposes.

As used in this application, “percent identity” between amino acid or nucleotide sequences is synonymous with “percent homology,” which can be determined using the algorithm of Karlin and Altschul (Proc. Natl. Acad. Sci. USA 87, 2264-2268, 1990), modified by Karlin and Altschul (Proc. Natl. Acad. Sci. USA 90, 5873-5877, 1993), or other methods. The noted algorithm is incorporated into the NBLAST and XBLAST programs of Altschul et al. (J. Mol. Biol. 215, 403-410, 1990). BLAST nucleotide searches are performed with the NBLAST program, score=100, wordlength=12, to obtain nucleotide sequences homologous to a polynucleotide of the invention. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (Nucleic Acids Res. 25, 3389-3402, 1997). When utilizing BLAST and Gapped BLAST programs, the default parameters of the respective programs (e.g., XBLAST and NBLAST) are used.

Nucleic acid and amino acid sequences of known C2 domains (e.g., those of synaptotagmins) can be used as a “query sequence” to perform a search against public databases to identify homologues, isoforms, or variants of wild-type C2 domains. Such searches can be performed using the NBLAST and XBLAST programs. BLAST nucleotide searches can be performed with the NBLAST program, score=100, wordlength=12 to obtain nucleotide sequences homologous to reference nucleic acid molecules. BLAST protein searches can be performed with the XBLAST program, score=50, wordlength=3 to obtain amino acid sequences homologous to the reference protein. To obtain gapped alignments for comparison purposes, Gapped BLAST can be utilized as described in Altschul et al., Nucleic Acids Res., 25:3389-3402, 1997. When utilizing BLAST and Gapped BLAST programs, the default parameters of the respective programs (e.g., XBLAST and NBLAST) can be used.

Whether a polypeptide or protein specifically binds to PtdS can be readily determined by a skilled artisan using a quantitative, semi-quantitative, or qualitative assay. These assays typically involve a binding target that contains PtdS, which can be artificial phospholipid vesicles, preparations of cellular phospholipid membranes (e.g., from lysed bacteria or erythrocytes), whole cells which express externalized PtdS on the cell surface (e.g., mammalian cells stimulated to induce apoptosis using chemicals or activated erythrocytes and platelets), or in vivo models which are known to contain apoptotic cell population (e.g., responsive tumors after chemotherapy or radiation treatments and apoptosis induced in other organs, including but not limited to the liver, thymus, heart, and muscles).

For the detection or quantification of binding, one can use labeled investigative protein or its derivative (e.g., by fluorescent dyes or radioisotopes). The binding affinity can be documented in terms of association constant (Ka) or dissociation constant (Kd). As an alternative, the protein itself is unlabeled and its binding is quantified using fluorescence quenching, surface plasmon resonance analysis, or immunochemistry with antibodies that recognize epitopes on the C2 domain or as part of C2 domain derivatives. As another alternative, unlabeled investigative protein can be “pulled down” using PtdS containing lipid vesicles or PtdS coated solid materials that can be readily separated from an aqueous phase using filtration or centrifugation. The presence of the investigative protein associated with PtdS-containing materials, accompanied by the reduction or absence of the investigative protein in the supernatant or aqueous phase, can be assessed using SDS-PAGE, immunochemistry staining, optical detection methods, or the detection of radioactive decays.

PtdS binding specificity and affinity can also be assessed with competition assays against a protein with known PtdS binding properties, such as annexin V or the C2A domain of synaptotagmin I. Such assays can be performed using artificial phospholipid membranes, cellular membrane preparations, whole cells, or in vivo targets with exposed PtdS. The assays examine the ability of the investigative protein to compete or prevent the binding of an established PtdS binding protein. Either or both proteins may be labeled for detection purposes, and the relative binding affinity can be assessed in terms of inhibitory concentration (IC₅₀) values.

Any radionuclide (used interchangeably with the term “radioisotope”) that is recognized as being useful for injection into a mammalian animal, preferably a human being, for nuclear imaging can be used to label a C2 domain or an active variant thereof. Examples of such radionuclides include but not limited to carbon 11, fluorine 18, gallium 67, gallium 68, indium 111, indium 113m, iodine 122, iodine 123, iodine 124, iodine 125, iodine 131, nitrogen 13, oxygen 15, technetium 99m (^{99m}Tc), and thallium 201. ^{99m}Tc is a preferred radionuclide for the purpose of the present invention. It is well within the capability of a skilled artisan to label a polypeptide with a radioisotope. In the case of labeling a polypeptide with ^{99m}Tc, the polypeptide is preferably thiolated first to enhance labeling.

Pharmaceutically acceptable carriers and vehicles can be used to form a composition or pharmaceutical formulation including the radionuclide-labeled compound described herein. The composition may be administered to a subject in an amount effective, at a dosage and for a period of time necessary, to achieve desired imaging result. An effective amount of the composition of the invention may vary according to factors such as animal species, age, body weight, and route of administration. An effective amount is also one in which any toxic or detrimental effects (e.g., side effects) of the composition are outweighed by the diagnostically beneficial effects. The dosage typically takes into consideration the amount of polypeptide injected and the amount and type of radionuclide injected. The compositions of the invention may be administered at a concentration of, for example, 1-1000 μg polypeptide/kg (body weight), 1-900 μg polypeptide/kg, 1-800 μg polypeptide/kg, 1-700 μg polypeptide/kg, 1-600 μg polypeptide/kg, 1-500 μg polypeptide/kg, 1-400 μg polypeptide/kg, 1-300 μg polypeptide/kg, 1-200 μg polypeptide/kg, 1-100 μg polypeptide/kg, 5-100 μg polypeptide/kg, 5-80 μg polypeptide/kg, 5-60 μg polypeptide/kg, 5-40 μg polypeptide/kg, or 5-20 μg polypeptide/kg.

^{99m}Tc can be administered to adult humans at doses up to about 20 mCi. The preferred dose for a single ^{99m}Tc administration is between about 3 and about 20 mCi.

Radionuclide-labeled C2 domain or an active variant thereof can be administered by any of several systemic and topical routes known to be effective for administration of radiolabeled proteins for nuclear imaging. For example, a composition of the present invention can be administered orally, parenterally, by inhalation, topically, rectally, nasally, buccally, vaginally, or via an implanted reservoir. The term “parenteral administration” includes subcutaneous, intravenous, intramuscular, intradermal, intrasternal, intraperitoneal, intrapleural, intralymphatical, intrahepatic, intralesional, and intracranial injection or infusion techniques. The compositions can also be administered via catheters or through a needle to any tissue. Methods for practicing the modes of administration listed above are known in the art.

A preferred method of administration is intravenous (i.v.) injection. It is particularly suitable for imaging of well-vascularized internal organs, such as the heart, blood vessels, and tumors. Methods for i.v. injection of radiopharmaceuticals are known. For example, it is recognized that a radiolabeled pharmaceutical is typically administered as a bolus injection using either the Oldendorf/Tourniquet method or the intravenous push method (see e.g., Mettler and Guierbteau, Essentials Of Nuclear Medicine Imaging, Second Edition, W. B. Saunders Company, Philadelphia, Pa., 1985).

For imaging a brain tumor, a composition of the present invention can be administered intrathecally. Intrathecal administration delivers compound directly to the sub-arachnoid space containing cerebral spinal fluid (CSF).

Imaging can be carried out using any suitable imaging device such as a gamma ray detector (e.g., a gamma scintillation camera or a 3-dimensional imaging camera) or by positron emission tomography (PET) or single photon emission computed tomography (SPECT). To facilitate interpretation of an image obtained, the image may be digitally processed to filter out background, noise and/or non-specific localization. In a preferred embodiment, imaging is carried out at about 0.5 hour to about 24 hours, about 0.5 hour to about 15 hours, about 0.5 hour to about 8 hours, about 0.5 hour to about 5 hours, about 2 hours to about 24 hours, about 2 hours to about 15 hours, about 2 hours to about 8 hours, about 2 hours to about 4 hours, about 3 hours to about 24 hours, about 3 hours to about 15 hours, about 3 hours to about 10 hours, about 3 hours to about 8 hours, about 3 hours to about 6 hours, or about 3 hours after administration of the radionuclide-labeled compound.

Under the present invention, a kit for in vivo imaging of cell death or another condition characterized by an increase in the extracellular level of PtdS in a mammal can be provided. The kit can contain a radionuclide-labeled compound comprising a C2 domain or an active variant thereof as described herein and an instruction on administering the compound to a mammal for imaging cell death or said condition.

The invention will be more fully understood upon consideration of the following non-limiting examples.

### EXAMPLE 1

### ^{99m}Tc Labeling of the C2A Domain of Synaptotagmin I as a Molecular Probe for Apoptotic and Necrotic Cell Death

This example demonstrates that C2A-GST specifically recognizes apoptotic and necrotic cells. When labeled with ^{99m}Tc, the radiotracer has relatively high radiochemical yield and purity, with the PtdS-binding activities of the C2A well preserved. The example also demonstrates non-invasive visualization of cell death using the above molecular probe.

Materials and Methods

Overexpression of C2A-GST protein: The fusion protein of C2A-GST, encoded in pGEX plasmid, was overexpressed in E. Coli bacteria (strain BL21) and purified as described in Zhao M et al. Nat. Med. 7:1241-1244, 2001, with minor modifications. A 50 ml overnight culture was used to inoculate 1 liter of Terrific Broth, in the presence of 0.1 mg/ml ampicillin. The culture was grown at 37° C. for 1 hour and protein expression was induced by adding 1 ml of isopropylthiogalactoside stock water solution to a final concentration of 0.1 mM. After another 3-hour growth at 37° C., the bacterial cells were collected by centrifugation at 5,000 g for 10 min at 4° C. The bacterial cell pellet was resuspended in 10 ml of lysis buffer (50 mM Tris, 200 mM NaCl, 5% glycerol, pH 7.4), and the cells were treated with one cycle of freeze-and-thaw. To degrade the bacterial cell wall, lysozyme was added to a final concentration of 0.3 mg/ml, and incubated for 40 min at room temperature. DNase was then added to a final concentration of 0.1 mg/ml, and incubation was continued for 30 min at room temperature. The bacterial lysate was centrifuged to remove insoluble materials at 10,000 g for 15 min at 4° C. The supernatant was loaded onto an affinity chromatography column with 5 ml bed volume of glutathione agarose (Sigma) pre-equilibrated with 20 mM Tris-HCl and 100 mM NaCl at pH 7.4. After extensive washing with the same buffer, until the elute had an absorbance of less than 0.02 at 280 nm, the C2A-GST fusion protein was eluted with 40 ml of 15 mM reduced form of free glutathione in the same buffer. The eluted fractions (5 ml each) that contained the fusion protein were pooled and dialyzed overnight with a 10 kDa molecular weight cut off membrane, in phosphate buffered saline (PBS, pH 7.4), freeze-dried and stored at −20° C. Protein purity was assessed by 10% sodium dodecyl sulfate polyacrylamide gel electrophoresis (SDS-PAGE), and the C2A-GST fusion protein appeared to be a single band upon visual inspection. The typical yield was 40 to 50 mg of the fusion protein from each liter of bacterial culture.

Fluorescent labeling of C2A-GST protein: C2A-GST-FITC was produced by adding 2.33 μl of FITC stock (12 mg/ml) in DMSO to 300 μl of C2A-GST (2 mg/ml) solution in PBS, and gently mixing for 3 hours at room temperature. The reaction was quenched with the addition of 100 μl of 1 M Tris buffer, pH 8.9. The fluorescently labeled protein was purified using gel filtration column chromatography (Sephadex G-25 Fine, 10 ml bed volume) equilibrated with PBS. Protein and FITC concentration of the elute fractions was monitored using a spectrophotometer (model UV 1601PC) at 280 and 495 nm, respectively. Protein concentration was determined using the Bradford method with BSA as standards. The FITC-to-protein molar ratio of the purified product was calculated to be greater than 0.9.

C2A-GST-Alexa Fluor 680 conjugate was synthesized by incubating C2A-GST (2 mg/ml) and Alexa Fluor 680 (Molecular Probes, 62.5 μg/ml) in borate buffer (25 mM borate, 100 mM NaCl, pH 8.2) for 3 hours at room temperature, with gentle mixing. The fluorescent protein product was purified by gel filtration as described above. Using this conjugation method, the dye-to-protein molar ratio was determined to be above 0.8.

Flow cytometry: Jurkat cells (ATCC, USA) were cultured in RPMI 1640 medium (Invitrogen) containing 10% fetal bovine serum (Gibco), at 37° C. with humidified environment and 5% CO₂. Cell viability was determined using trypan blue dye and was found to be greater than 95%. Apoptosis was induced by incubating 5×10⁶ cells/ml with 3.5 μmol/L final concentration of camptothecin (Sigma) for 5 hours. By the end of the incubation period, typical morphological appearances of apoptosis have become apparent, including cell shrinkage, blebbing and some apoptotic body formation.

For flow cytometry analysis, treated and untreated cells were harvested by centrifugation at 600 g for 5 min, washed with cold RPMI 1640 medium for 3 times, and adjusted the cell concentration to 10⁶/mL with cold binding buffer (2 mM CaCl₂, 15 mM HEPES, 120 mM NaCl, pH 7.4). Three microliters of C2A-GST-FITC and 3 μl of propidium iodide (50 mM) were added to 500 μl of cell suspension and incubated for 5 min. The samples were analyzed using a Becton Dickinson (San Jose, Calif.) FACScan flow cytometer. Identical procedure was repeated using FITC-Annexin V (Molecular Probes).

For direct comparison on cell binding activity between C2A-GST and Annexin V, double-labeling was achieved by co-incubating treated cells with C2A-GST-Alexa Fluor 680 (red, excitation 680 nm, emission 710 nm) and Annexin V-FITC (green, excitation 490 nm, emission 515 nm). For each flow cytometry analysis, 10,000 cells were counted.

^{99m}Tc labeling of C2A-GST: ^{99m}Tc labeling C2A-GST was prepared as follows. 200 μl of C2A-GST (2 mg/ml) solution in PBS was mixed with 2.96 μl of 2-iminothiolane (10 mg/ml, Sigma) in DMSO (Fisher). The mixture was gently shaken for 5 min at room temperature, and placed at 37° C. for 1 hour. For 99mTcO₄-labeling protein, 500 μl 99mTcO₄-(about 5 mci) in 0.9% NaCl was added to a stannous glucoheptonate (GH) mixture (80 μg stannous chloride and 8 mg sodium glucoheptonate), and the reaction was kept at room temperature for 10 min under N₂. 400 μl of ^{99m}Tc-GH solution was mixed with 2-IT modified C2A-GST and incubated for 30 min at room temperature. Radiochemical purity of the product was analyzed using ITLC-SG chromatographic strips (Gelman Sciences) with saline as mobile phase.

The degree of thiolation after 2-iminothiolane modifications was estimated using Ellman's reagent. Specifically, 100 μl of thiolated C2A-GST (as described above) was purified by gel filtration to remove unreacted 2-iminothiolane, using a 5 ml bed volume of Sephadex G25 Fine, equilibrated with PBS, pH 7.4. Fractions of 200 μl were eluted with PBS, and the presence of protein was monitored with absorbance at 280 nm. The fractions that contained the protein peak were pooled. 50 μl of the protein solution was mixed with 50 μl of Ellman's reagent at 0.01 M, in 0.1 M phosphate buffer, pH 8.0. After 15 min incubation at room temperature, the absorbance at 412 nm was measured. A calibration curve was constructed using absorbance values obtained using L-Cys as a sulfhydryl standard. In parallel, protein concentration in the solution was determined using the Bradford method, as specified by the manufacture (BioRad).

Gel filtration chromatography (Sephadex G-25, column PD-10, Amersham Biosciences, Sweden) equilibrated with PBS, pH 7.4, was performed to separate 99mTc labeled C2A-GST from free 99mTc pertechnetate. The labeled protein was eluted with PBS at a flow rate of 1 ml/min. Protein concentration was determined using the Bradford method (BioRad, according to manufacturer's protocol), with absorbance at 512 nm. The stability of the labeled protein was measured by leaving aliquots of 99mTc-C2A-GST in saline at room temperature, and determining the presence of free pertechnetate after designated time intervals, at 1, 2, 4, 6 and 8 hours, by instant thin-layer chromatography. For control purpose, bovine serum albumin was labeled with ^{99m}Tc in an identical protocol as described above.

K_{d} measurement and competition assay: The binding affinity of ^{99m}Tc-C2A-GST to apoptotic cells was assessed in terms of dissociation constant (K_{d}), using a saturation method. Non-specific and specific binding groups were prepared using healthy Jurkat cells and those treated with camptothecin for 5 hours, respectively. The apoptotic index was determined for each according to flow cytometric analysis. While less than 2% of the cell population were non-viable in the untreated cells, the rate of apoptosis was 25% in the campothecin treated sample. 50 μl cell suspensions at a density of 10⁷/ml were incubated with increasing final concentration of ^{99m}Tc-C2A-GST from 3.98 nM to 510 nM for 10 min at room temperature with gentle agitation. To separate free from bound protein, the reaction mixture was centrifuged at 1,000 g for 1 min through 100 μl silicon oil, where the aqueous solution remained above the silicon oil layer and the cells were collected at the bottom. After the supernatant and oil layers were removed, the tips of the tubes that contained the cell pellet were cut off and their radioactivity was measured. The values of specific binding were obtained by subtracting the radioactivity of non-specific binding tubes from binding tubes. The scattered plot was obtained with specific binding in counts per min (cpm) as Y axis, and the protein concentrations of ^{99m}Tc-C2A-GST in nM as X axis. After drawing the best-fit saturation curve and determining the maximum binding (Bₘₐₓ), Kd was estimated as the concentration of ^{99m}Tc-C2A-GST at one half of maximum binding (B_{1/2}). The final K_{d} was calculated as an average of the three independent measurements, with standard deviations.

The binding specificity of ^{99m}Tc-C2A-GST relative to the unlabeled C2A-GST was investigated with a competition assay. In this assay, 50 μl 10⁷/ml apoptotic cells were incubated with 20 μl ^{99m}Tc-C2A-GST (final concentration 78 nM) in the presence of increasing concentrations of unlabeled C2-GST varied from 15.9 nM to 7,830 nM for 10 min at room temperature with gentle agitation. Bound and free protein was separated using silicon oil as described above. The percentage of radioactivity remains bound to the apoptotic cells was calculated, and plotted against the concentration of unlabeled C2A-GST. The inhibitory concentration (IC₅₀) at this particular experimental condition was estimated to be the concentration of unlabeled protein required to reduce the specific binding of the radiolabeled protein by 50% (B50).

Specific uptake in acute myocardial infarction in rats: The animal study conformed to the institutional and national Guide for the Care and Use of Laboratory Animals, with institutional approval. Sprague-Dawley rats, with body weight between 300 to 390 g, were anesthetized with sodium pentobarbital (50 mg/kg) intraperitoneally, and respiration was maintained using a rodent ventilator. Real time electrocardiogram (ECG) was monitored throughout the surgery and imaging times. Thoracotomy was performed and the chest was opened at the fourth intercostal space to expose the heart. The pericardium was opened with forceps, and a 6.0 suture was passed underneath the left anterior descending coronary artery at the level between the pulmonary trunk and the left atrium. Coronary occlusion was achieved by tightening the suture. The success of occlusion was confirmed by the pale appearance in the area at risk and the immediate changes in ECG profiles, including a significant broadened QRS complex, and elevation of ST segment. In this model, ventricular tachycardia generally occurred at 7 to 15 min of ischemia and the Q wave depressed gradually throughout the whole procedure. After 30 min of ischemia, reperfusion was initiated by cutting the suture.

^{99m}Tc-C2A-GST (0.2 mCi per rat) was injected via a femoral vein catheter at 90 min after reperfusion, and blood samples were collected from femoral artery on the lateral side at designated time points (0.5, 1, 2, 3, 5, 10, 15, 20, 25, 30, 40, 50 and 60 min). After 1 hour, the animal was euthanized and the heart was removed and rinsed with 15 mM HEPES buffer. Pieces of myocardial tissues were collected from the infarcted and remote viable regions, weighed and countered for radioactivity using a gamma counter (LKB WALLAC 1282 COMPU GAMMA). This protocol was repeated using NHS-acetate-inactived ^{99m}Tc-C2A-GST as control to measure non-specific uptake. The radioactivity uptake in the myocardium was expressed as % ID/g tissue.

Results

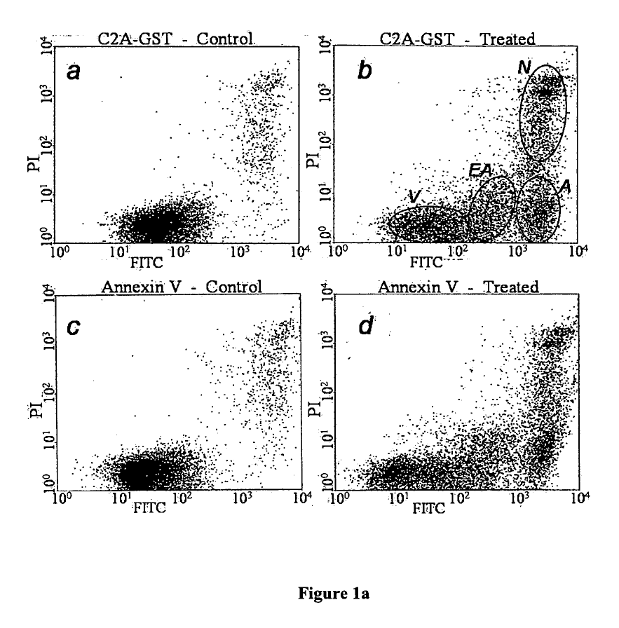

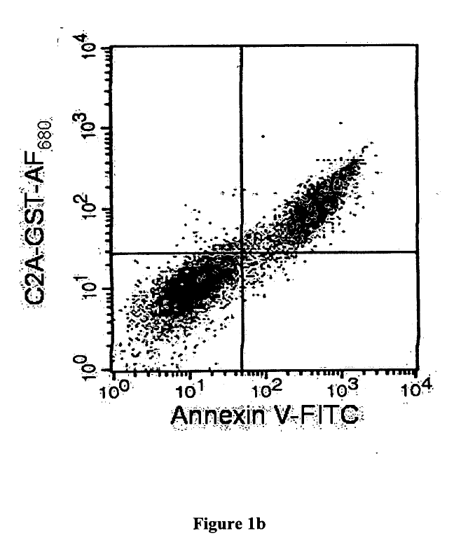

FITC labeling C2A-GST and flow cytometry: After FITC labeling, the average fluorescin-to-protein molar ratio of the purified conjugate was estimated to be 1.22±0.53. FIG. 1a shows the flow cytometry profile of camptothecin-treated Jurkat cells labeled with C2A-GST-FITC and PI. According to the fluorescent intensity difference, four distinct cell populations are clearly identifiable. These include viable (V), necrotic (N), apoptotic (A) and early apoptotic (EA). Flow cytometric analysis using a commercially available Annexin V kit yielded near identical distribution among treated Jurkat cells. Double-labeling using red fluorescent C2A-GST-AF680 and green fluorescent Annexin V-FITC produced either double-positive or double-negative cells, demonstrating that both molecular probes recognized the same individual cells (FIG. 1b). The red and green fluorescence intensity is dependent on the ratios between C2A-GST-AF680 and Annexin V-FITC concentrations.

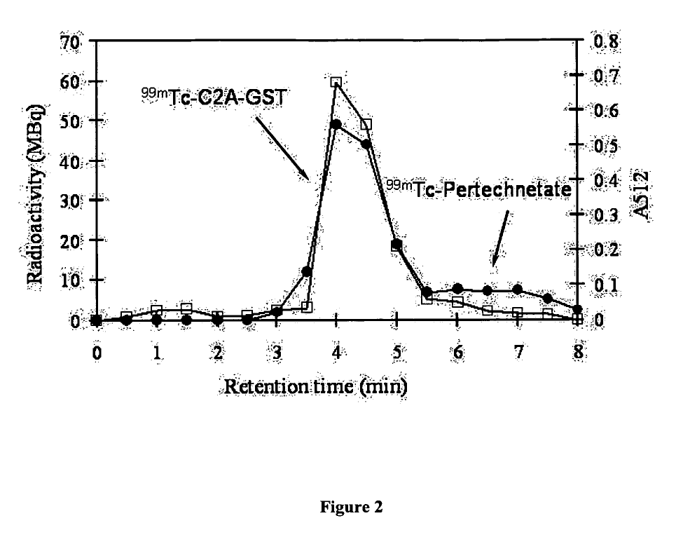

^{99m}Tc labeling of C2A-GST: The C2A-GST fusion protein was labeled with ^{99m}Tc to relatively high radiochemical yield and purity, following thiolation with 2-iminothiolane. The optimum degree of thiolation, as determined using Ellman's reagent, was 7 sulfhydryl groups per protein molecule. At this ratio, the radiochemical purity of ^{99m}Tc-C2A-GST was 82.3% and 95.5%, before and after size exclusion purification, respectively. The elution profile from size exclusion chromatography is shown in FIG. 2. The protein peak co-registers with the peak of radioactivity, with a retention time of 4 min. Free ^{99m}Tc-pertechnetate has a longer retention time, at 6.5 min. The radiochemical purity of labeled C2A-GST was also confirmed to be greater than 95% using instant thin-layer chromatography on silica gel. The specific radioactivity of gel filtration purified ^{99m}Tc-C2A-GST was estimated to be 20-30 μCi/μg protein. The non-thiolated C2A-GST fusion protein showed no significant incorporation of radioactivity (data not shown). The stability of the labeled protein was tested in saline at room temperature. Over an 8-hour period, the protein retained the radiolabel, as determined by instant thin-layer chromatography (Table 2).

TABLE 2
Stability of ^{99m}Tc-C2A-GST in vitro

| | Time (hr) | | | | | |
|---|---|---|---|---|---|---|
| | 0 | 1 | 2 | 4 | 6 | 8 |
| Radiochemical purity (%) | 95.45 | 95.20 | 94.58 | 93.69 | 92.87 | 91.57 |

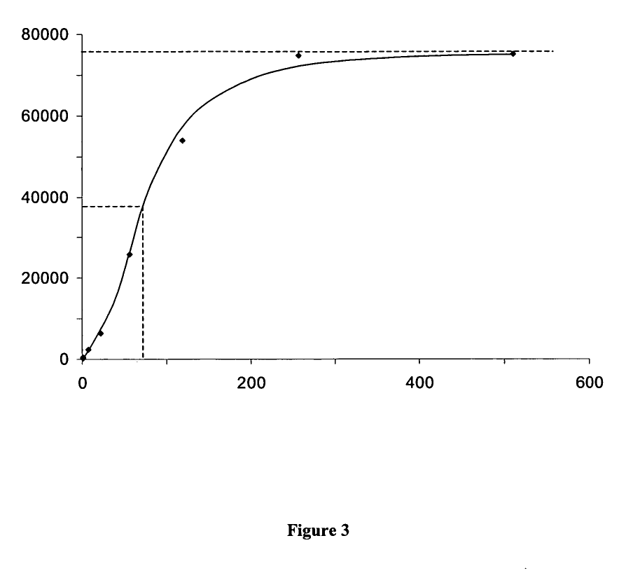

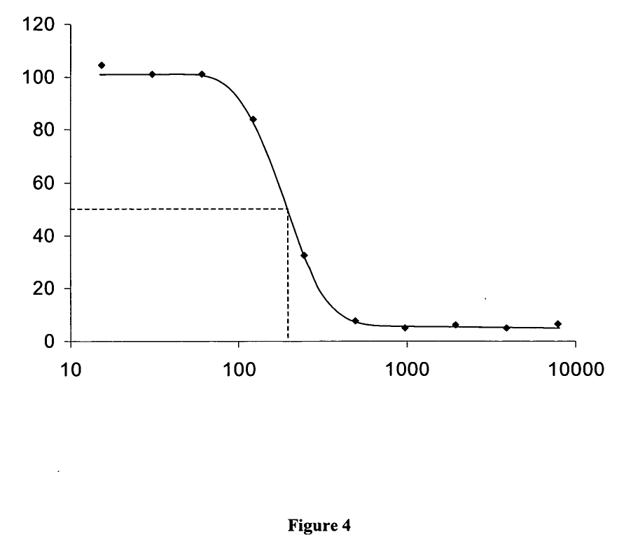

Binding affinity and specificity: The estimation of Kd of ^{99m}Tc-C2A-GST toward apoptotic cells was obtained using a saturation binding assay. A representative saturation curve is shown in FIG. 2, demonstrating the interaction of the radiolabeled protein with a finite number of binding sites in these cells. At half maximal binding, the Kd was determined to be 7.10±1.48×10⁻⁸ M (FIG. 3). The binding of ^{99m}Tc-C2A-GST was reversible in the presence of unlabeled competing C2A-GST, with an IC₅₀ of 17.4±0.84×10⁻⁸ M at the current experimental conditions (FIG. 4).

In vivo studies in animal model of acute myocardial infarction: The pharmacokinetics of ^{99m}Tc-C2A-GST in the rat appears to be bi-phasic, with reasonably fast blood clearance. The blood half-life of the fast phase is estimated to be 15 min. At 1 hour after injection, about 20% of the injected dosage remained in circulation. The radioactivity in the myocardial infarct area was about 23-27 fold higher than that of non-infarct area. However, in the animal model injected with inactive ^{99m}Tc-C2A-GST, there was only a 2-6 fold difference between infarct area and non-infarct area.

In summary, the results here demonstrate that C2A can be used to recognize apoptosis and necrosis and the C2A-GST fusion protein can be radiolabeled without significantly altering the above function. Therefore, a radiolabeled C2A domain such as ^{99m}Tc-C2A-GST can be used as an imaging probe to non-invasively detect cell death such as myocardial cell death in acute infarction.

Fluorescently labeled C2A-GST binds to cells in different modes and stages of cell death. The uptake of C2A-GST-FITC is distinct in necrotic and apoptotic cells. An intermediate population was also identified with significant C2A-GST-FITC uptake, but negative for PI. These individuals appear to be at a transitional state toward fully-developed apoptosis and necrosis, with externalized PtdS and intact plasma membrane integrity. This observation was also confirmed using fluorescently labeled Annexin V. Results from the double-staining experiment using fluorescent C2A-GST and Annexin V indicate that the two proteins interact with the same groups of cells. The fact that the uptake of Annexin V is dependent on the presence and concentration of C2A-GST and vice versa indicates that the two proteins compete for a finite number of common binding site.

There are 4 native cysteine residues in the primary structure of C2A-GST. Thiolation of the C2A-GST fusion protein greatly enhanced radiolabeling of the protein. The lack of technetium ^{99m}Tc incorporation prior to 2-iminothiolane modification indicates that the endogenous thiol groups may not form favorable chelating sites for the radioisotope. After thiolation, the subsequent radiolabeling procedure can be completed within 30 min at room temperature. This standard protocol means that the labeling could be performed at a typical nuclear imaging facility. Using the current method, the labeled C2A-GST has relatively high radiochemical purity, yield and good radiostability. At the end of an 8-hour stability test conducted at room temperature, the radiochemical purity declined less than 4%.

As PtdS is a molecular marker for both apoptosis and necrosis, the binding of PtdS holds the key to the utility of ^{99m}Tc-C2A-GST as an imaging agent targeted to cell death. The binding activity of this radiotracer was quantitatively evaluated in terms of binding affinity apoptotic cells, where the Kd was estimated to be 7.1×10⁻⁸ M.

After radiolabeling, ^{99m}Tc-C2A-GST appeared to have preserved its specificity, as evident from the fact that the binding of ^{99m}Tc-C2A-GST was reversible in the presence of unlabeled competing C2A-GST. Such observation suggests that both forms of C2A-GST specifically interact with the same well-defined binding targets in apoptotic cells.

Results from in vivo imaging experiments indicate that ^{99m}Tc-C2A-GST, as a target-specific molecular probe, is effective in the non-invasive detection of acute myocardial infarction. Focal uptake of radioactivity in tomographic images were positively correlated to high levels of irreversible cellular damages within the injured coronary territories, as identified in histological analysis. The accumulation of radiolabeled C2A-GST to the infarct regions may be attributed to the binding of exposed PtdS.

### EXAMPLE 2

### Imaging Myocardial Cell Death in the Acute Phase of Infarction with ^{99m}Tc Labeled C2A Domain of Synaptotagmin I

This example demonstrates the non-invasive imaging of myocardial apoptosis at 3 hr post injection with single photon emission computed tomography (SPECT), using an acute infarction pig model.

Materials and Methods

Induction of acute infarction: Animal procedures were carried out strictly following NIH guidance and with institutional approval. Adult pigs (20 kg, n=7) were initially anesthetized with intramuscular injection of katamine hydrochloride (10 mg/kg), and remained anesthetized with I.V. dosages of propofol (1% sulution, 6 mg/kg per hour). Each animal was intubated and mechanically ventilated, with its heart rate and profile constantly monitored with electrocardiography (EKG). After installation of a catheter sheath in the right femoral artery, a 6F catheter was delivered into the coronary artery. The localization of the catheter, and the patency of the downstream coronary artery branches were confirmed with x-ray angiography. Arterial occlusion was initiated by inserting and inflating an angioplasty balloon at one of the following locations: distal end of the left descending coronary artery (n=1), circumflex branch (n=5), or right coronary artery (n=1). Ischemia typically lasted between 20-30 min, and reperfusion was initiated by deflating the angioplasty balloon. Both coronary occlusion and the subsequent reperfusion in each animal were confirmed by x-ray angiography. Meanwhile, EKG profiles typical of acute infarction were recorded, including ST segment elevation and gradual Q wave formation.

Radiolabeling of C2A-GST. The fusion protein of C2A-GST was overexpressed in E. Coli, purified and labeled with ^{99m}Tc as described in in example 1. Briefly, C2A-GST was first thiolated with 2-iminothiolane to an average of 7 sulfhydral groups per protein. After removing unreacted 2-iminothiolane with gel filtration, C2A-GST was labeled with ^{99m}Tc using a stannous glucoheptonate solution. The radiochemical purity of ^{99m}Tc-C2A-GST was confirmed to be greater than 95% using size exclusion chromatography and instant thin layer chromatography. The specific activity of the radiotracer was generally between 20-30 μCi/μg protein. Binding assays using lysed blood cell membranes indicated that the binding affinity and specificity of ^{99m}Tc-C2A-GST was not significantly altered compared with unlabeled C2A-GST.

Biodistribution: Animal procedures were conducted following NIH guidance and with institutional approval. Healthy male and female C57 black mice (64 total, 8-10 weeks old) were divided randomly into 8 groups. Each mouse was injected with ^{99m}Tc-C2A-GST (0.74 MBq) via the tail vein. At each time point of 1, 15, 30, 60, 120 and 240 min post injection, one group of mice were sacrificed by cervical dislocation. The uptake of radioactivity was measured for the blood, heart, liver, spleen, lung, kidney, stomach, intestines, muscle and bone, by gamma counting with energy levels set between 120 and 170 keV. The data were expressed as a percentage of injected dose±standard deviation (ID %±Stdv).

In vivo imaging of myocardial cell death: Within 1 hour after reperfusion, ^{99m}Tc-C2A-GST (6-7 mCi/animal) was injected I.V. and SPECT and CT images were acquired with a Millennium VG Hawkeye dual modality SPECT/CT scanner (General Electric), at 3 hour post injection. After CT scans covering the chest cavity (40 axial slices, 15 second each), SPECT data were acquired at energy peak of 140 KeV, window of 20%, matrix size of 128×128 and 60 angle views at 6° each counted for 45 seconds. A subgroup of 5 animals were sacrificed after 3 hours and each heart was removed for histological analysis. The remaining animals were subject to imaging again at 6 and 17 hour post injection, and sestamibi perfusion imaging. For blood half-life measurement, venous blood samples of 0.1 ml were withdrawn from each animal (n=7) at 10 min intervals for the first 3 hours after injection of the radiotracer. The radioactivity of all blood samples was determined with scintillation counting and corrected for radioactive decay.

Ex vivo measurement of radiotracer uptake: At the end of the 3-hour imaging session, the animal was sacrificed. The heart was removed, drained and quickly rinsed with saline to remove excessive blood. The infarct regions could be identified downstream of the affected coronary artery branch as pale areas upon visual inspection. Tissue samples from the infarct regions and healthy remote regions were removed, weighted and counted for radioactivity with scintillation counting. The results were expressed as count per minute (cpm) per gram tissue, with a mean value and standard deviation.

Ex vivo assessment of myocardial cell death: To identify characteristic ultrastructural changes of myocardial cell death, representative myocardial fragments from the infarct and remote viable regions were fixed in 4% paraformaldehyde and 0.5% glutaraldehyde for 24 h at 4° C. The tissues were dehydrated, sectioned and mounted on carbon grids. Transmission electron micrograph was acquired with magnification of 64,000 times.

In addition, flow cytometric analysis was performed on these tissue specimens to quantitatively assess the presence of cells with sub-G₀ DNA content.

Results

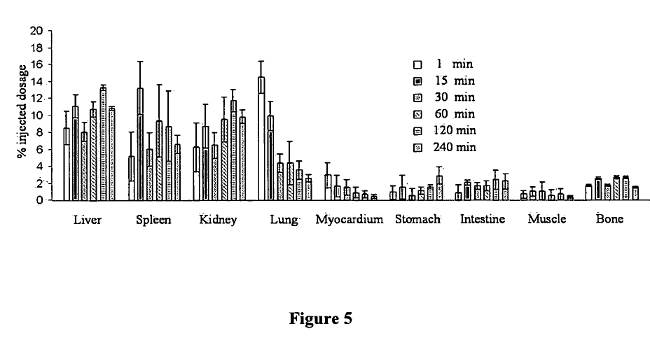

Biodistribution in mice: Radioactivity uptake as a function of time in different tissues is summarized in FIG. 5. Following the injection of the radiolabeled C2A-GST, there was rapid uptake by the liver and kidneys. By 120 min, the ID % in the liver and kidneys were 13.2±4.3 and 11.7±2.5, respectively, indicating clearance by the hepatic/biliary and the urinary systems. Blood clearance was reasonably fast, with less than 15% of the initial value after 120 min, and with low uptake in the myocardium. The presence of radioactivity in the spleen and lung was significant, while that in skeletal muscle was negligible. In all dosages of ^{99m}-Tc-C2A-GST tested in pigs and mice, no sign of toxicity or significant adverse effect was observed.

SPECT imaging: SPECT and CT images were obtained from individual animals with coronary occlusion at the left circumflex and left anterior descending branch, respectively. The images were acquired at 3 hours post injection of ^{99m}Tc-C2A-GST. We observed, in co-registered SPECT and CT images, significant focal uptake of the radiotracer at the posterior wall of the left ventricle. A high level of radioactivity was also detected at the apex region, consistent with the ischemia/reperfusion injury inflicted by the occlusion of the left anterior descending arterial branches. In all cases, with SPECT/CT co-registration, the high uptake of radioactivity in the myocardium, rather than in the ventricular blood pool, was clearly resolved. The signal-to-background ratio between infarct and remote viable myocardium was 3.36±0.74. The image quality from later time points, at 6 and 17 hours, indicated that the signal-to-background ratio continues to improve slightly and became plateaued by 17 hours (data not shown). The locations of high radioactivity uptake were not shifted over time, but the signals appeared to become less diffused and more focal. Perfusion defects identified with ^{99m}Tc-sestamibi SPECT images correlated well with high uptake of ⁹⁹mTc-C2A-GST. Blood clearance profile of ^{99m}Tc-C2A-GST appeared to be bi-phasic. The blood half-life of the fast clearance phase was calculated to be around 15 min (n=7). As blood samples were withdrawn only for the first 3 hours post injection, more data from later time points would be necessary to accurately derive the blood half-life of the slow clearance phase. Nonetheless, this value could be estimated to be in the order of several hours. In addition to the infarct areas, there was significant radioactivity accumulation in the liver and the kidneys, consistent with previous biodistribution studies in mice.

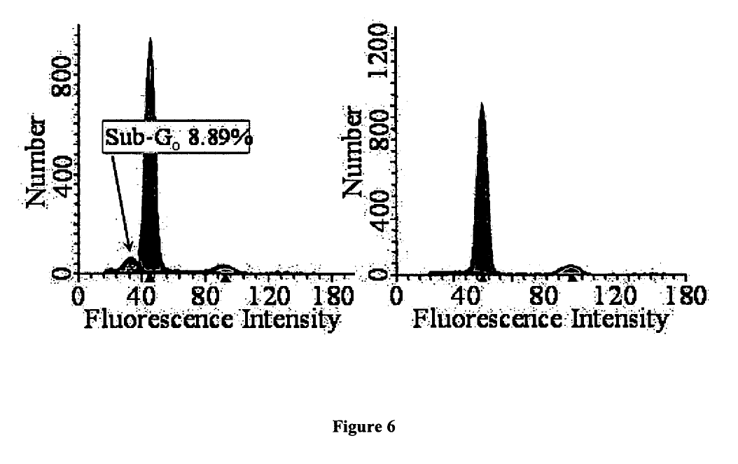

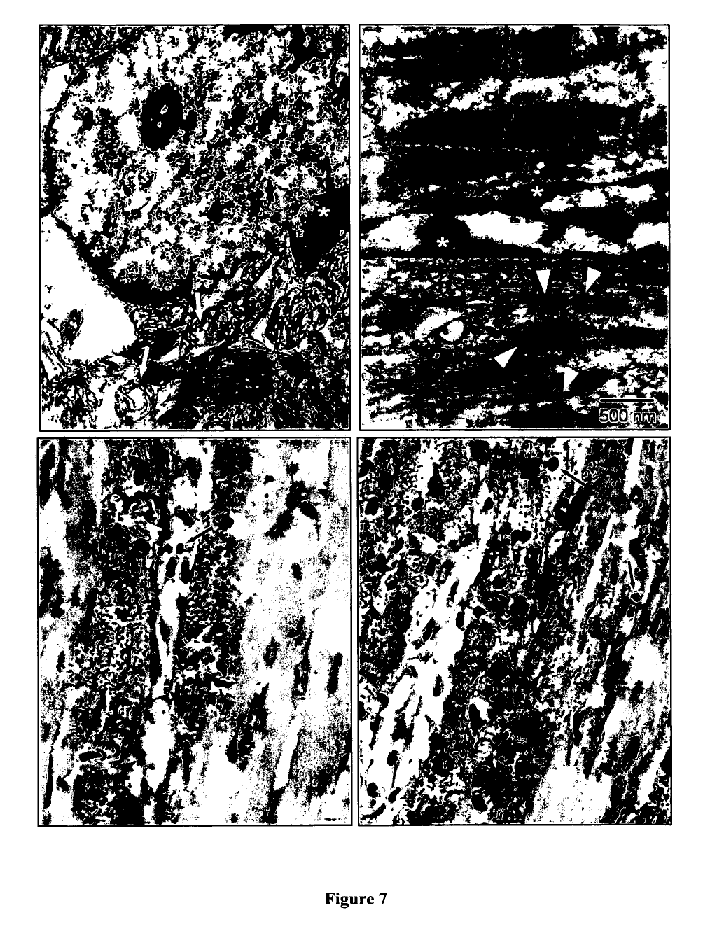

Tissue analysis: Postmortem analysis also confirmed the co-localization between radioactivity and injured myocardium: Scintillation counting of tissue samples revealed an 11.68±4.02 fold elevation in radioactivity uptake at the infarct region compared with remote viable myocardium. Flow cytometric analysis of the infarct tissues indicated that 8.9% of the total cell population are at sub G₀ phase, with significantly reduced DNA content (FIG. 6). On the other hand, remote healthy myocardium had cell death rate at below 0.1%. (FIG. 6). Transmission micrograph documented the occurrence of both apoptosis and necrosis at the site of infarct, including chromatin condensation and the swelling of mitochondria, respectively (FIG. 7).

The present invention is not intended to be limited to the foregoing examples, but encompasses all such modifications and variations as come within the scope of the appended claims.

We claim:

1. A method for detecting cell death or another condition characterized by an increase in the extracellular level of phosphatidylserine in a mammalian subject in vivo comprising the steps of:
(a) administering a radionuclide-labeled compound that comprises a phosphatidylserine-binding C2 domain of a protein or an active variant thereof; and
(b) measuring radiation emission from the radionuclide in the subject to obtain an image of radiation emission,
wherein the site of said cell death or condition can be determined from the image.

2. The method of claim 1 wherein the method is for detecting myocardial infarction, a vascular thrombus, an arthrosclerosis plaque, or tumor cell death.

3. The method of claim 1 wherein the method is for detecting acute myocardial infarction.

4. The method of claim 1, wherein the subject is selected from the group consisting of a human being, a pig, a rat, and a mouse.

5. The method of claim 1, wherein the subject is a human being.

6. The method of claim 1, wherein the cell death is apoptotic or necrotic cell death.

7. The method of claim 1, wherein radionuclide is selected from the group consisting of carbon 11, fluorine 18, gallium 67, gallium 68, indium 111, indium 113m, iodine 122, iodine 123, iodine 124, iodine 125, iodine 131, nitrogen 13, oxygen 15, technetium 99m, and thallium 201.

8. The method of claim 1, wherein the radionuclide is technetium 99m.

9. The method of claim 1, wherein the C2 domain is thiolated for technetium 99m labeling.

10. The method of claim 1, wherein the C2 domain is selected from the group consisting of human synaptotagmin I C2A domain, pig synaptotagmin I C2A domain, rat synaptotagmin I C2A domain, and mouse synaptotagmin I C2A domain.

11. The method of claim 1, wherein the C2 domain is rat synaptotagmin I C2A domain.

12. The method of claim 1, wherein the active variant is at least 60% identical to the phosphatidylserine-binding C2 domain.

13. The method of claim 1, wherein the active variant is at least 70% identical to the phosphatidylserine-binding C2 domain.

14. The method of claim 1, wherein the active variant is at least 80% identical to the phosphatidylserine-binding C2 domain.

15. The method of claim 1, wherein the active variant is at least 90% identical to the phosphatidylserine-binding C2 domain.

16. The method of claim 1, wherein the radiation detector is a gamma ray detector and the radiation emission is gamma ray emission.

17. The method of claim 1, wherein the radiation detected by positron emission tomography or single photon emission computed tomography.

18. The method of claim 1, wherein the radionuclide-labeled compound is administered intravenously.

19. The method of claim 1, further comprising the step of repeating step (b) at selected intervals wherein the repeating is effective to track changes in the intensity of radiation emission in the subject over time to detect changes either in location or in number of cells that undergo cell death.

20. A kit comprising:
a radionuclide-labeled compound comprising a phosphatidylserine-binding C2 domain of a protein or an active variant thereof; and
instruction on administering the compound into a mammalian subject to image cell death or another condition characterized by an increase in the extracellular level of phosphatidylserine.
